(19) United States
(12) Patent Application Publication
(10) Pub. No.: US 2025/0339076 A1
(43) Pub. Date: Nov. 6, 2025

(54) FLEXIBLE BIOELECTRODE DEVICE AND METHOD OF MANUFACTURING A FLEXIBLE BIOELECTRODE DEVICE

(71) Applicant: Body Biosense Pty Ltd, Newport NSW (AU)

(72) Inventor: Jett Van Der Wallen, Newport NSW (AU)

(73) Assignee: Body Biosense Pty Ltd, Newport NSW (AU)

(21) Appl. No.: 19/265,820

(22) Filed: Jul. 10, 2025

Related U.S. Application Data
(63) Continuation of application No. PCT/AU2023/050769, filed on Aug. 15, 2023.

(30) Foreign Application Priority Data
Jan. 16, 2023 (AU) 2023900083

(51) Int. Cl. A61B 5/266; A61B 5/259; A61N 1/04

(52) U.S. Cl. CPC A61B 5/266; A61B 5/259; A61N 1/0476; A61N 1/0496; A61B 2560/0468; A61B 2562/046; A61B 2562/125; A61B 2562/16; A61B 2562/227

(57) ABSTRACT

The invention has been developed primarily for use in/with sensing biological signals and/or stimulating a body part of a subject. The invention relates to flexible bioelectrode(s) device and a method of manufacturing such a flexible bioelectrode device. The flexible bioelectrode device comprises a moulded body, a wire array of conductive wires at least partially embedded in the moulded body; the wire array defining terminal ends and distal ends; wherein each of the terminal ends of the wire array are connected to an electrode; and at least one or more connector terminals connected to the distal ends of the wire array.

### CROSS-REFERENCE TO RELATED PATENT APPLICATIONS

This Patent Application is a Continuation of co-pending PCT Patent Application No. PCT/AU2023/050769, filed Aug. 15, 2023, which is now pending, the entire teachings and disclosure of which are incorporated herein by reference thereto.

This Patent application claims priority to Australian Provisional Patent Application No. 2023900083, filed Jan. 16, 2023, the entire teachings and disclosure of which are incorporated herein by reference thereto.

### FIELD OF THE INVENTION

The present invention relates to flexible bioelectrode(s) device.

The invention has been developed primarily for use in/with sensing biological signals and/or stimulating a body part of a subject and will be described hereinafter with reference to this application. However, it will be appreciated that the invention is not limited to this particular field of use.

### BACKGROUND OF THE INVENTION

A (biopotential) electrode in a bioelectrical system detects or transmits ionic currents, by the transduction of electric currents. Biopotential electrodes can be part of therapeutic or diagnostic devices. These devices can be surface, transcutaneous, or implantable.

Surface devices measure bioelectrical signals via surface electrodes, superficial to the skin. Transcutaneous devices have electrodes that penetrate the skin. Implantable devices have electrodes underneath the skin. For example, an electrocardiogram (ECG) device uses electrodes to sense the electrical activity of the heart. This can be for detecting an ECG waveform. Another example is a myoelectric sensor (also known as electromyogram or EMG), which uses electrodes to sense the electrical activity in muscles for rehabilitation and diagnosis of musculoskeletal health and disease.

A bioelectrical sensor or stimulator typically comprises 5 hardware components: (1) Electrodes; (2) Wires (if required); (3) Circuit connector; (4) Electrical circuit and data transmission device and (5) Connection to computer.

Electrodes can be wet, semi-dry or dry, and can be surface, transcutaneous, or implantable. Wet electrodes have a sensing/stimulating interface which comprises a gel or liquid, which aids conduction. Electrodes can be resistive or capacitive, based on whether the interface is conductive or insulating.

Electrodes can be polarisable or non-polarisable, based on whether they form a charged electrode-electrolyte double layer. The amplitude of surface bioelectrical signals is in the range of 0-10 mV, requiring amplification of approximately 1000-fold for volt-range data acquisition to a computer, for sensing applications.

Data transmission to the computer can be wired or wireless. The computer can record or transmit these signals via computer memory, a cloud server, or a portable storage device.

It is desirable to be able to monitor or stimulate the bioelectrical signals of a human or animal subject anywhere. This can be for diagnostic or rehabilitation purposes. There is a demand for user-friendly and robust sensing or stimulating devices.

There are challenges associated with sensing or stimulating bioelectrical signals, whether surface, transcutaneous or implantable. Firstly, the electrode must have good physical contact with a particular body part of the subject, from which the bioelectrical signal is being transmitted or received. This is for the duration of the sensing or stimulating period.

Secondly, the electrodes must be comfortable.

Thirdly, the electrodes must be sufficiently robust to withstand movement of the subject while signals are being received or transmitted.

Prior art electrodes used in sensing or stimulating devices comprise a layered configuration. Rapid prototyping technologies (RPTs) can be utilised to fabricate the electrodes or wires on a substrate layer. These can be bottom-up technologies such as 2D printing, 3D printing, vapour deposition, coating, casting, or moulding. Alternatively, they can be top-down technologies such as chemical, mechanical, laser or plasma cutting and etching. The wires are typically printed on a substrate and then covered with an insulating layer, such that they do not interface with the body part.

The wires are typically connected to electrical circuitry via a connector.

One of the disadvantages of prior art electrodes and wires, is when they are subject to movement such as bending and flexing, the layers can delaminate. Furthermore, the wires can be prone to breaking at any discontinuities such as connection points to electrodes and connectors.

For example, surface electrodes can be rigid. When pressed against the body part to ensure good skin contact, the electrodes can cause discomfort and lead to pressure sores over time. In the case of implantable electrodes, rigid electrodes can cause a foreign body reaction, due to a mechanical mismatch between hard electrodes and soft tissue.

The present invention seeks to provide a solution, which will overcome or substantially ameliorate at least some of the deficiencies of the prior art, or to at least provide an alternative.

It is to be understood that, if any prior art information is referred to herein, such reference does not constitute an admission that the information forms part of the common general knowledge in the art, in Australia or any other country.

### SUMMARY OF THE INVENTION

According to an aspect of the present invention, a method of manufacturing a flexible bioelectrode device is provided, the method comprising the steps of:
a. inserting a wire array defining terminal ends for termination at an electrode and distal ends for connection to a connector into a first mould portion;
b. partially enclosing the wire array in a mould while exposing the terminal ends and the distal ends;
c. moulding a fluid prepolymer or polymer around the wire array to form an embedded wire array, leaving distal ends and terminal ends of the wire array exposed.

The method may further include the step of fabricating a wire array on a permeable polymeric substrate.

The method may further include the step of affixing an electrode on each of the terminal ends of the wire array.

The step of affixing an electrode may comprise fabricating a conductive substrate and a sensing/stimulating interface of the electrode at a terminal end of the wire array

The wire array and electrodes may be fabricated together in a single operation.

The method may further include the step of attaching at least one or more connectors to distal ends of the wire array.

Each sensing/stimulating interface of the electrode may be surface treated to aid with sensing or stimulating of bioelectrical signals.

The method may include the step of applying a conductive liquid, gel or adhesive to the sensing/stimulating interface.

The step of applying a conductive liquid, gel or adhesive may include applying an applicator sheet to the moulded flexible bioelectrode device, exposing the sensing/stimulating interface of each of the electrodes.

In another aspect of the present invention, a flexible bioelectrode device is provided comprising:
a. a moulded body;
b. a wire array of conductive wires at least partially embedded in the moulded body, the wire array defining terminal ends and distal ends;
c. wherein each of the terminal ends of the wire array are connected to an electrode; and
d. at least one or more connector terminals connected to the distal ends of the wire array.

The wire array of conductive wires may be fabricated using a rapid prototyping method.

The flexible bioelectrode device may further comprise a permeable polymeric film onto which the conductive wires are fabricated.

The permeable polymeric film may be adapted to permit fluid prepolymer or polymer to permeate through the film when the film is in contact with fluid prepolymer or polymer.

The fluid prepolymer or polymer may be a thermoplastic or thermosetting polymer.

The thermoplastic polymer may be any one of the following but not limited to: Cellulose, Cellulose derivatives, Cyclic transparent optical polymer, Parylene, Polymethylmethacrylate, Polyamide (Nylon), Polybutylene terephthalate, Polycarbonate, Polyester, Polyethylene, Polyethylene terephthalate, Polyethylenimine, Polylactic acid (PLA), Polypropylene, Polystyrene, Polyvinyl alcohol (PVA), Styrene-ethylene-butylene-styrene, Thermoplastic polyurethane.

The thermosetting polymer may be any one of the following but not limited to: Latex, Polychloroprene, Polydimethylsiloxane (PDMS, Silicone), Polyimide (Kapton), Polyurethane.

The flexible bioelectrode device may comprise of a sensing/stimulating interface, conductive substrate, surrounded by an insulating base.

The flexible electrodes may be affixed when a conductive substrate of each electrode is deposited or coated onto a respective distal end of the wire array.

The flexible bioelectrode device may further include a conductive gel, liquid, or adhesive applied to an outer exposed surface of each sensing/stimulating interface of each electrode.

The conductive wires, conductive substrate, and/or sensing/stimulating interface of the flexible bioelectrode device may comprise a conductive element or compound, with or without a binder.

This invention may also be said broadly to comprise the parts, elements and features referred to or indicated in the specification of the application, individually or collectively, and any or all combinations of any two or more of said parts, elements or features, and where specific integers are mentioned herein which have known equivalents in the art to which this invention relates, such known equivalents are deemed to be incorporated herein as if individually set forth.

Other aspects of the invention are also disclosed.

### BRIEF DESCRIPTION OF THE DRAWINGS

Notwithstanding any other forms which may fall within the scope of the present invention, embodiments of the present invention will now be described, by way of example only, with reference to the accompanying drawings in which:

FIG. 1 is a flexible bioelectrode device in accordance with an embodiment of the present invention;

FIG. 2 is an exploded view of the flexible bioelectrode device in accordance with the embodiment shown in FIG. 1;

FIG. 3 illustrates a cross-section of an electrode of a flexible bioelectrode device in accordance with an embodiment of the present invention;

FIG. 4 illustrates an exploded view showing a mould and components for a flexible bioelectrode device in accordance with an embodiment of the present invention;

FIG. 5 illustrates an exploded view of a section of the mould and components for a flexible bioelectrode device;

FIG. 6 illustrates the components of the flexible bioelectrode device assembled within an open mould, and

FIG. 7 illustrates an applicator for applying a conductive gel, liquid or adhesive to a flexible bioelectrode device and a flexible bioelectrode device in accordance with an embodiment of the present invention.

### DESCRIPTION OF EMBODIMENTS

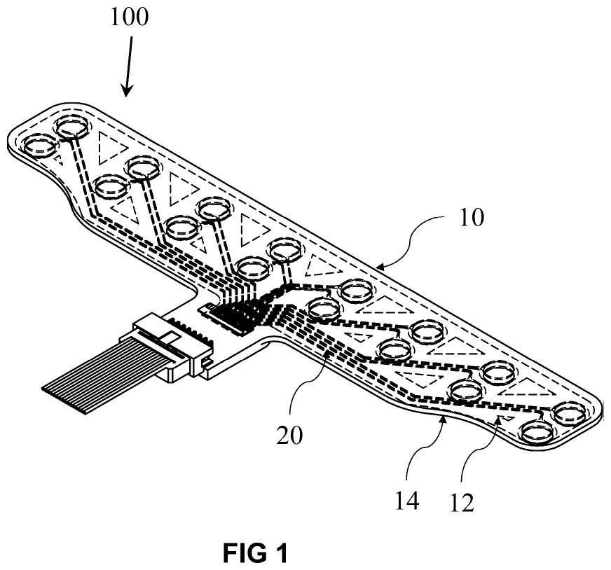

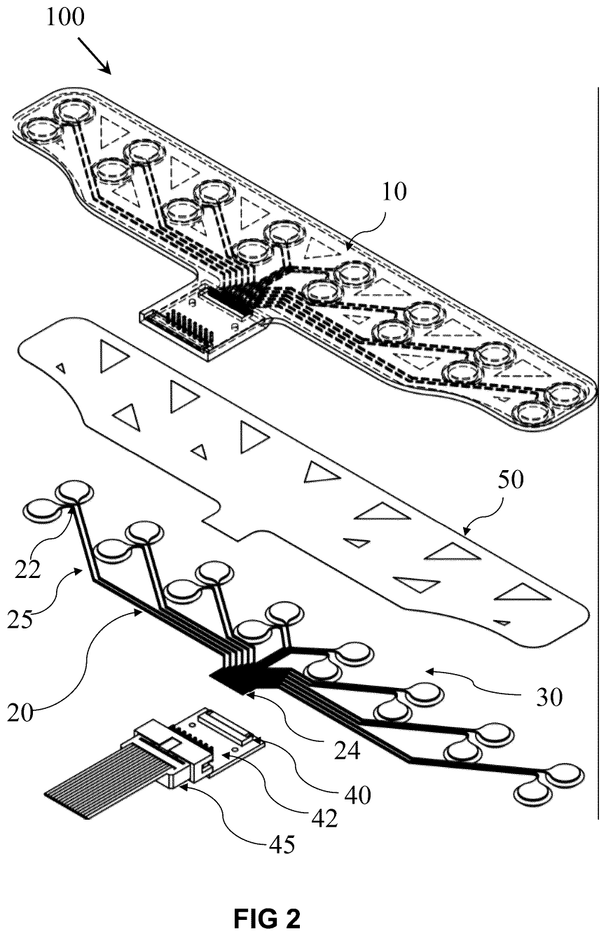

A flexible bioelectrode device according to a first aspect of the invention is generally indicated by the numeral 100 and shown in FIG. 1. An exploded view of the flexible bioelectrode device is shown in FIG. 2. The flexible bioelectrode device 100 comprises a moulded body 10. The flexible bioelectrode device 100 further comprises a wire array 20 of conductive wires at least partially embedded in the moulded body 10. The wire array 20 comprises terminal ends 22 and distal ends 24, shown in FIG. 2. Each of the terminal ends 22 of the wire array are connected to an electrode 30 and the distal ends 24 of the wire array are connected to at least one connector terminal 40.

In the illustrated embodiment in FIG. 1, the body 10 is elongated and substantially rectangular. It should be noted that the body can be of any shape or size. The body 10 has a first surface 12 and an opposed second surface 14. The first surface 12 and the second surface 14 are separated by a thickness. In this embodiment, the width is a couple of millimetres.

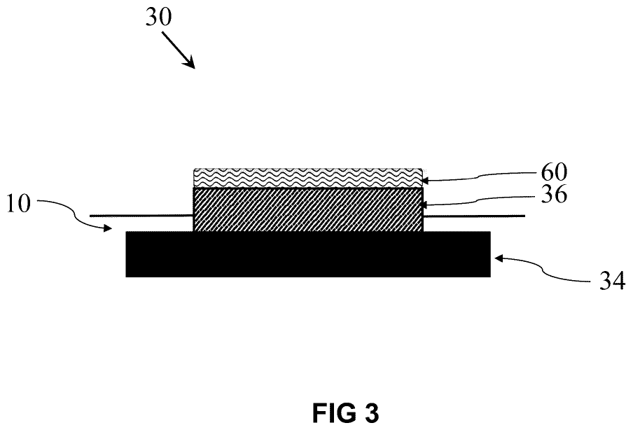

The second surface 14 has holes within which electrodes sit to allow for electrodes to be exposed and in contact with the body part. The first surface 12 is sealed. FIG. 3 shows a cross-section of an electrode. As described below in further detail and shown in FIG. 3, each electrode 30 has a sensing/stimulating interface 36 for contact with the body part. The sensing/stimulating interfaces 36 of the electrodes may be recessed, level or raised relative to the second surface 14.

The wire array 20 of conductive wires 25 can be fabricated onto a substrate or can be ready-made individual wires arranged in an array. In the illustrated embodiment, the wire array 20 is fabricated using rapid prototyping technologies. Advantageously, fabricating the wire array 20 with rapid prototyping technologies allows for complex designs and wire arrangements to be fabricated with precision. The wires will also take up less space. Rapid prototyping technologies are also fast, efficient, cost and time effective and minimize waste. Advantageously, the flexible bioelectrode device is re-usable, again minimizing fabrication time, cost, and waste.

FIG. 1 illustrates an example of a wire configuration within the flexible bioelectrode device in accordance with one embodiment.

The electrodes are linearly arranged along a length of the flexible bioelectrode device in two rows. It should be noted that the electrodes may be a single electrode (0-dimensional) or arranged in a multiform consisting of various shapes or sizes (1-dimensional, 2-dimensional, or 3-dimensional). The conductive wires extend between each electrode 30 and the connector terminal 40.

The flexible bioelectrode device 100 further comprises a permeable polymeric film 50 onto which the conductive wires 25 can be fabricated. The permeable polymeric film 50 supports the conductive wires 25 in position as they are being fabricated in the desired configuration.

The permeable polymeric film 50 is adapted to permit fluid prepolymer or polymer to permeate through the film 50 when the film 50 is in contact with fluid prepolymer/molten polymer. This allows the fluid to surround the wires on all sides such that when the fluid prepolymer/molten polymer cures, the conductive wires 25 are embedded within the cured polymer. In contrast to existing methods which use a layered approach, the risk of delamination of any layers is eliminated and movement of the wires is significantly reduced. This reduces the risk of breakage of the wires. Furthermore, the cured polymer band 10 is flexible and can conform closely to a body part without the conductive wires breaking. If fabricating the wires and electrodes from a conductive-polymer composite, the mechanical properties of the wires and electrodes are approximately equivalent to that of the insulating polymer. As the conductive wires, electrodes, and insulating polymer act as one cohesive piece, bending and flexing of the flexible bioelectrode device will likely not break the conductive components.

In an example, polydimethylsiloxane (PDMS) is used as a fluid prepolymer. The fluid prepolymer can be any suitable non-toxic and/or biocompatible prepolymer that cures into a flexible solid form. Upon curing at room temperature or catalysed by the application of heat, the fluid prepolymer becomes a solid polymer. Alternatively, it can be a molten polymer. The type of insulating polymer can be thermoplastic or thermosetting, depending on whether it can be reheated back to a molten state. Other examples of insulating polymers that are available in a fluid form (either prepolymer or molten polymer) are listed in the table below. The insulating polymer or prepolymer is not limited to the examples provided below.

The following list contains some examples of suitable/compatible insulating polymers:

List of insulating polymers

| Type | Examples |
|---|---|
| Thermoplastic | Cellulose, Cellulose derivatives, Cyclic transparent optical polymer, |
| | Parylene, Polymethylmethacrylate, Polyamide (Nylon), Polybutylene |
| | terephthalate, Polycarbonate, Polyester, Polyethylene, Polyethylene |
| | terephthalate, Polyethylenimine, Polylactic acid (PLA), Polypropylene, |
| | Polystyrene, Polyvinyl alcohol (PVA), Styrene-ethylene-butylene- |
| | styrene, Thermoplastic polyurethane |
| Thermoset | Latex, Polychloroprene, Polydimethylsiloxane (PDMS, Silicone), |
| | Polyimide (Kapton), Polyurethane |

As shown in FIG. 2 and FIG. 3, each electrode 30 comprises an insulating base 10, a conductive substrate 34 and a sensing/stimulating interface 36. The sensing/stimulating interface 36 and/or the conductive substrate 34 may or may not be fabricated at the same time and in the same manufacturing step as the conductive wires 25. If fabricated at the same time, this reduces differences in material properties and discontinuities between the components, which otherwise may cause breakage or delamination.

As mentioned above, the electrodes consist of a conductive substrate 34 and a sensing/stimulating interface 36 that interfaces with the body to sense or stimulate a bioelectrical signal. The insulating base 10 is provided by the cured polymer within which the electrodes 30 and wires 25 are embedded.

The conductive wires 25 extend from a first end 22 to a second end 24. The first end of each conductive wire is affixed to an electrode 30. The second end 24 is affixed to the connector terminal 40. The connector terminal 40 is adapted to connect to an internal electrical circuit 42. One such example of a connector terminal 40 is a zero-insertion force (ZIF) connector. Another example is a soldered connection. Other suitable connectors can also be used. If required, the internal electrical circuit 42 can connect to an auxiliary connector 45 which can be connected to an external electrical circuit (not shown). Signals received or transmitted by the electrodes can be transferred via the conductive wires to the terminal connector 40, and then to the internal electrical circuit 42. Either the internal or external electrical circuit can transmit data either wired or wirelessly to an external device, e.g., computer.

FIG. 3 shows a cross-section of an electrode. As mentioned previously, electrodes 30 are affixed when a sensing/stimulating interface 36 and conductive substrate 34 of each electrode 30 is fabricated onto a respective distal end 24 of the wire array 20. This fabrication can be done in one or many steps, creating a unibody design or a laminar design. The surface of the sensing/stimulating interface 36 can either be flat or patterned. Some examples include a microneedle, micropillar, microfiber, microparticle, microporous, or fractal design.

The flexible bioelectrode device 100 further comprises a conductive liquid, gel, or adhesive layer 60 applied to an outer exposed surface of each sensing/stimulating interface 36 of each electrode. This allows for enhanced electrical contact by decreasing the electrical impedance at the skin-electrode interface. The conductive liquid, gel, or adhesive layer 60 also helps reduce motion artefacts and helps to improve signal transduction. Conductive gels contain electrolytes (ions or ionic compounds), polymers, dispersion adhesive-like compounds, and water. Conductive liquids contain electrolytes dispersed in water, such as saline. Conductive adhesives contain ionic compounds and adhesive polymers/prepolymers.

A method of manufacturing the flexible bioelectrode device 100 will now be described.

The first step is to prepare the polymeric film 50. The polymeric film 50 can be surface treated to aid the adhesion of the conductive substrate 34. In one such instance, the first surface of the polymeric film is covered with a protective cover that is removable. A second surface opposite the first surface is removably covered with a non-stick paper. The polymeric film 50 can be manufactured to the desired size and shape, by any suitable cutting mechanism.

The polymeric film 50 is then aligned and attached to a fabrication bed. An adhesive can aid in the attachment of the underside of the polymeric film 50 to the fabrication bed.

If present, the protective cover is removed to expose the surface of the polymeric film 50.

By uploading a computer-aided design (CAD) file onto the fabricating machine, in accordance with the operating principles of the machine, conductive wires 25 and the conductive substrate 34 of the electrode are fabricated on the polymeric film 50. This can be via top-down or bottom-up fabrication methods. The sensing/stimulating interface 36 of the electrode can be fabricated at the same time or in a separate step, forming the electrodes 30. Several rapid prototyping technologies are listed in the below table, but is not limited to.

The following list contains some examples of suitable/compatible rapid prototyping technologies:

List of rapid prototyping technologies

| Type | Examples |
|---|---|
| 2D printing | Aerosol-jet printing, Inkjet printing, Doctor blade coating, Mayer bar |
| | coating, Roller printing, Screen printing |
| 3D printing | Fused deposition modelling (FDM) 3D printing, Liquid deposition |
| | modelling 3D printing, Stereolithography 3D printing |
| Deposition | Surface micromachining, Chemical vapour deposition, Physical vapour |
| | deposition, Drop casting, Micromoulding, Open moulding, Dip coating, |
| | Electroplating, Immersion plating, Electrospinning, Spin coating, Spray |
| | coating, Syringe extrusion, Surface-initiated atom transfer radical |
| | polymerisation |
| Etching | Bulk micromachining, Copper etching, Cutting, CNC routing, Laser |
| | cutting, Mechanical cutting, Plasma oxygen etching, Dissolving |
| Textile | Embroidery, Knitting, Sewing, Weaving |
| Joining | Gluing, Hot stamping, Lamination, Chemical lamination, Thermal |
| | lamination |
| Curing | Annealing, Drying, Freeze drying, Reaction sintering, Sintering |
| Particle orientation | Electrophoresis, Magnetic field orientating |
| Surface treatment | Acidification, Molecular adhesive, Oxygen plasma, Ozone, Ultraviolet |
| | (UV) |

One such method is FDM 3D printing, wherein components are printed layer-by-layer, forming a 3-dimensional body upon completion.

In the illustrated embodiment, the electrodes are fabricated by CAD-design and rapid prototyping technologies. The conductive wires, conductive substrate and/or sensing/stimulating interface comprises a conductive material, with or without an insulating binder. The table below illustrates different examples of conductive materials. The conductive materials are not limited to this list. The aforementioned insulating polymer table illustrates different examples of insulating polymers that can function as binders, but is not limited to. One such composite material is polylactic acid (PLA) doped with carbon conductive particles. The PLA is insulating while the carbon is conductive. As a whole, the material is conductive.

The following list contains some examples of suitable/compatible conductive materials:

List of conductive materials

| Type | Examples |
|---|---|
| Metal | Aluminium, Brass, Chromium, Copper, Galinstan, Gallium, Gold, |
| | Indium, Nickel, Nitinol, Platinum, Silver, Silver chloride, Stainless-steel, |
| | Tin, Titanium, Zinc, Zinc oxide |
| Intrinsically | Poly(3,4-ethylenedioxythiophenene):Polystyrene sulfonate, Poly(3,4- |
| conductive | ethylenedioxythiophenene), Polypyrrole, Poly(3-hexylthiophene) |
| polymer (ICP) | |
| Carbon | Carbon black, Graphene, Graphene oxide, Graphite, Multi-walled carbon |
| | nanotubes, Single-wall carbon nanotubes |

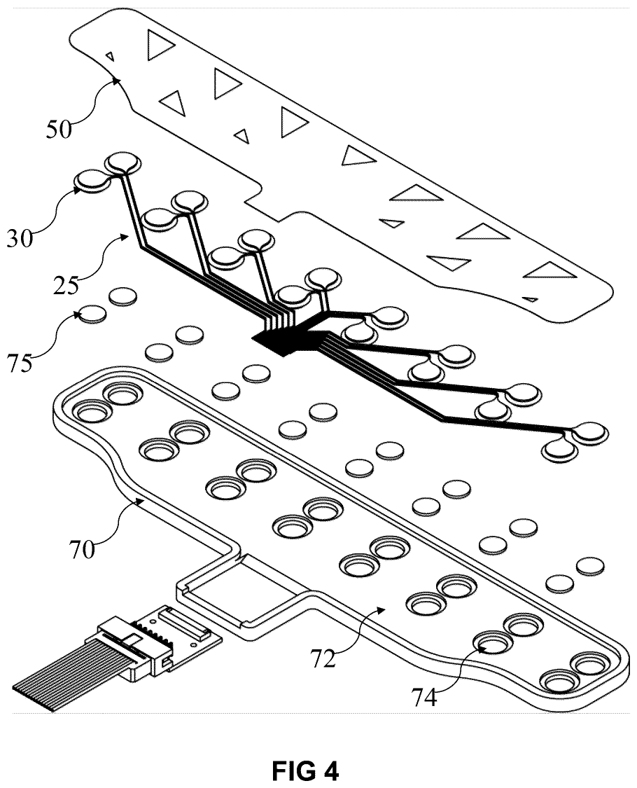

The method further comprises fabricating an open mould 70, which can be surface treated to aid with the removal of the flexible bioelectrode device 100. FIG. 4 illustrates an exploded view of the components of the flexible bioelectrode device 100 and mould 70. The mould 70 can be designed using CAD software. The open mould 70 can be fabricated via several different methods. This can be either bottom-up or top-down fabrication.

The mould 70 can be made of a suitable moulding material that is able to withstand temperature and pressure of the moulding process without structurally deforming. The mould 70 can also be made of a material that does not chemically react with the fluid prepolymer/molten polymer. One example of bottom-up fabrication is a 3D-printed polymer mould that has a non-stick surface. One example of top-down fabrication is a Computer Numerical Control (CNC)-machined stainless-steel mould.

The mould surface 72 must be made non-stick so that it does not stick to the polymer and to allow easy removal of the cured/solidified flexible bioelectrode device 100. In one such example, the mould surface 72 is sprayed with polytetrafluoroethylene (PTFE) spray, which is non-stick after it dries. In another example, the mould surface 72 is micromachined to achieve a smooth surface.

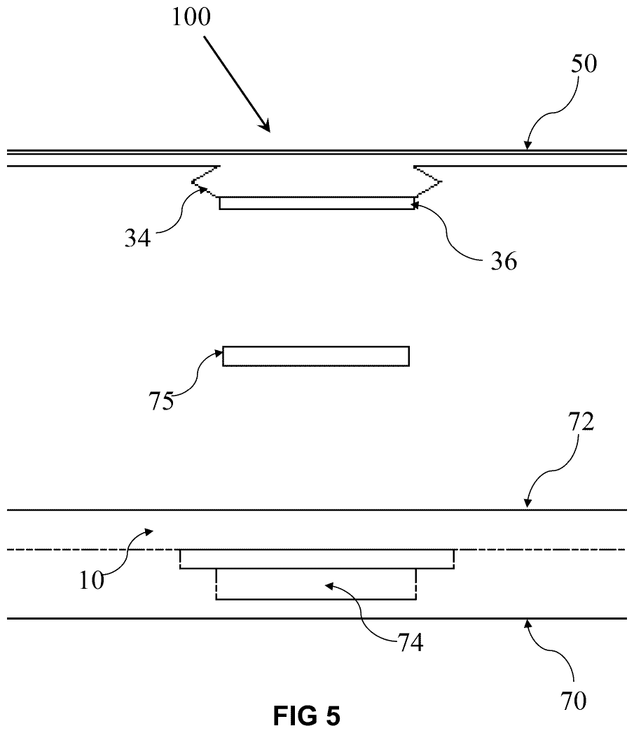

The next step is to assemble the printed wires in the mould 70, as per FIGS. 4 and 5. The mould 70 defines areas 74 for the electrodes 30. These areas 74 comprise recesses for the electrodes, which may be of any shape and size. A filler or spacer 75 is inserted into each of the recesses. This filler or spacer 75 prevents the fluid prepolymer/molten polymer from contacting the sensing/stimulating interface 36 of the electrodes 30, when the fluid prepolymer/molten polymer is poured into the mould 70.

FIG. 5 shows an exploded view of a recess, filler and an electrode.

In the embodiment, the polymer film 50 with the fabricated electrodes 30 and wires 25 is aligned and positioned within the mould 70 such that the first surface is in contact with the mould surface 72. The fabricated electrodes 30 are then pushed into their corresponding recess above each filler or spacer 75. The connector terminal 40 is aligned with the second or terminal ends of the wires 22 and connected to the wires. If present, the polymeric film backing is then peeled off the polymeric film 50. The filler or spacer 75 may or may not be required, depending on whether the sensing/stimulating interface 36 is recessed.

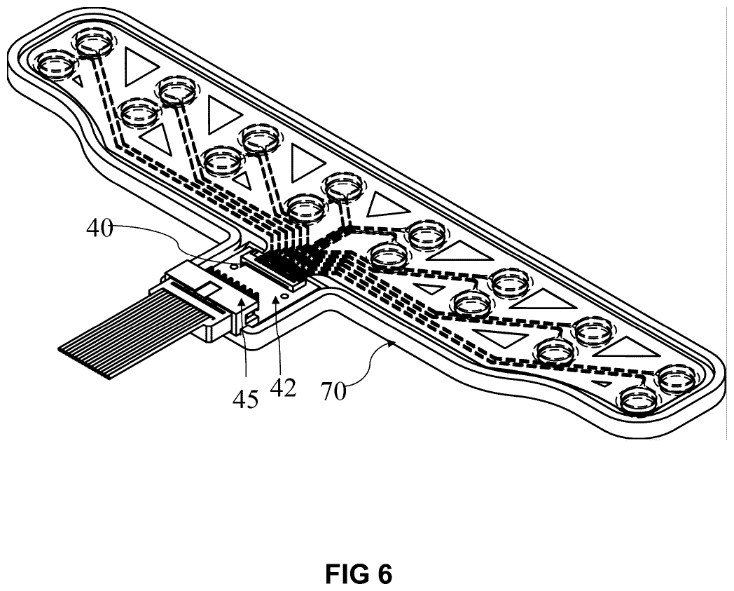

FIG. 6 shows all components assembled within the open mould.

The method further comprises preparing and adding the fluid prepolymer/molten polymer. In one such example, the fluid prepolymer is prepared by mixing the base of PDMS prepolymer with the curing agent. This mixture is uniformly mixed. The mixture is then degassed, removing any gases.

This mixture is then poured over the mould 70 including the polymer film with the wires 25 and electrodes 30.

The top surface of the poured prepolymer/molten polymer is smoothed with a squeegee or scraper, so that the fluid prepolymer/molten polymer is evenly distributed through the mould and permeates the recesses.

For molten polymers, the mixture is left to cool, such that it solidifies to a flexible polymer. In the case of a fluid prepolymer mixture, the mixture is cured by a catalyst (time, heat and/or electromagnetic radiation such as ultraviolet light). Depending on the nature of the prepolymer, different catalysts can be used. For room temperature (approximately 22 degrees Celsius) curing, approximately 48 hours is required for the prepolymer to convert fully to the flexible polymer. At elevated temperatures of 100 degrees Celsius or above, the time required is generally less than 1 hour. For ultraviolet curing, a UV lamp is generally situated less than 1 metre away, and can cure in a few minutes, at approximately 100 mW/cm2 intensity.

A scalpel can aid in the removal of the flexible bioelectrode device 100 from the mould 70. The flexible bioelectrode device 100 can be lifted out of the mould and further cured by a catalyst, if required.

In another embodiment, there are no recesses 74 for the electrodes in the mould. Instead, the insulating base 10 is etched away at the locations of the electrodes 30, leaving the sensing/stimulating interface 36 exposed.

The method further comprises applying conductive gel, liquid or adhesive 60 to the exposed sensing/stimulating interface 36 of the electrodes 30.

The method comprises designing and fabricating an applicator sheet 90.

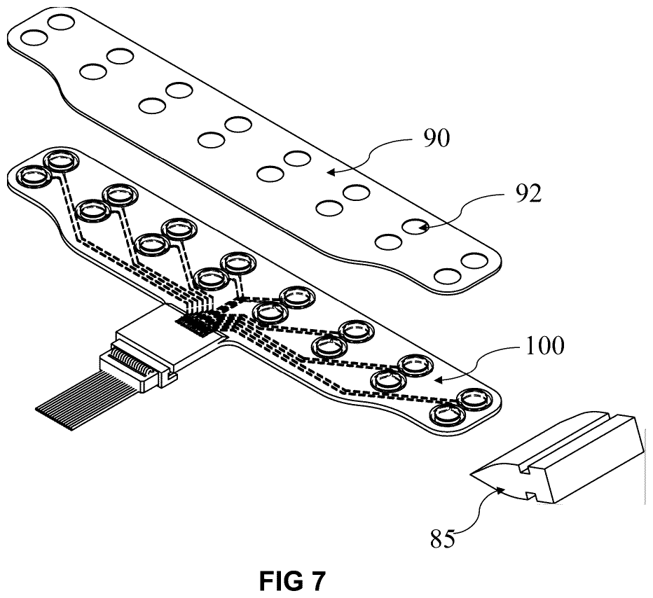

The applicator sheet 90 has holes 92 arranged across the width and length of the sheet, as illustrated in FIG. 7. Each hole 92 corresponds to the location of an electrode 30 in the flexible bioelectrode device 100. This is so that conductive gel, liquid or adhesive 60 can be selectively applied to the surface of the exposed sensing/stimulating interface 36 of the electrode 30. If a dry electrode surface is desired, no conductive gel, liquid or adhesive 60 will be applied to the surface of each electrode.

The flexible bioelectrode device 100 is placed on a surface with the conductive interface 36 side up. The applicator sheet 90 is aligned so that the holes 92 align with the electrodes 30 and then is placed on top of the flexible bioelectrode device 100. Conductive gel, liquid or adhesive 60 is spread across the applicator sheet 90 using a squeegee or scraper 85, penetrating into the holes 92 as shown in FIG. 7.

After the conductive gel, liquid or adhesive 60 is applied, the applicator sheet 90 is carefully removed without removing the conductive gel, liquid or adhesive 60.

The flexible bioelectrode device 100 is then removed. If an external electrical circuit is desired, this can be achieved via the connector 45. In another embodiment, the flexible bioelectrode device 100 is configured such that the flexible bioelectrode device 100 can communicate wirelessly with an external electrical circuit or device, via the internal electrical circuit 42.

The flexible bioelectrode device 100 is then applied to a body part. In one such example, this can be a limb, with or without the conductive gel, liquid or adhesive 60 contacting the body part. When sensing or stimulating signals via the skin, when using the conductive gel, liquid or adhesive 60, removal of hair is not always necessary.

For a surface or transcutaneous device, the flexible bioelectrode device can be secured in place to a body part with a bandage, garment, or other such methods.

Advantageously, the flexible bioelectrode device can be re-used. If used, the conductive gel, liquid or adhesive can be removed and re-applied on the electrode surfaces, to retain good signal quality. As this flexible bioelectrode device is mechanically robust, it can be used while moving to sense or stimulate bioelectrical signals in real-time. This allows for multiple sensing or stimulating of electrical signals across the device. The electrodes are embedded within the cured polymer, increasing the mechanical and electrical robustness of the device.

### EMBODIMENTS

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly, it should be appreciated that in the above description of example embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description of Specific Embodiments are hereby expressly incorporated into this Detailed Description of Specific Embodiments, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

### Comprising and Including

In the claims which follow and in the preceding description of the invention, except where the context requires otherwise due to express language or necessary implication, the word “comprise” or variations such as “comprises” or “comprising” are used in an inclusive sense, i.e., to specify the presence of the stated features but not to preclude the presence or addition of further features in various embodiments of the invention.

Any one of the terms: including or which includes or that includes as used herein is also an open term that also means including at least the elements/features that follow the term, but not excluding others. Thus, including is synonymous with and means comprising.

### Scope of Invention

Thus, while there has been described what are believed to be the preferred embodiments of the invention, those skilled in the art will recognize that other and further modifications may be made thereto without departing from the spirit of the invention, and it is intended to claim all such changes and modifications as fall within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

Although the invention has been described with reference to specific examples, it will be appreciated by those skilled in the art that the invention may be embodied in many other forms.

### INDUSTRIAL APPLICABILITY

It is apparent from the above, that the arrangements described are applicable to industries where sensing and/or stimulating using electrodes is desired.

What is claimed is:

1. A method of manufacturing a flexible bioelectrode device, the method comprising the steps of:
providing an internal electrical circuit, the internal electrical circuit comprising a connector;
providing a wire array, the wire array comprising:
one or more terminal end(s), each one or more terminal end(s) terminating at a respective electrode; and
one or more distal end(s), each one or more distal end(s) configured to
electrically connect to the connector;
electrically connecting the one or more distal end(s) to the internal electrical circuit via the connector to provide an electrically connected internal electrical circuit and wire;
providing a mould;
inserting the electrically connected internal electrical circuit and wire array into the mould;
providing a fluid prepolymer or polymer;
moulding the fluid prepolymer or polymer around the electrically connected internal electrical circuit and wire array by a single moulding process to provide an at least partially embedded electrically connected internal electrical circuit and wire array; and
allowing the fluid prepolymer or polymer around the at least partially embedded electrically connected internal electrical circuit and wire array to cure and thereby at least partially encapsulate the electrically connected internal electrical circuit and wire array in a unibody polymeric body to provide the flexible bioelectrode device.

2. The method of manufacturing a flexible bioelectrode device according to claim 1, wherein the wire array is fabricated on a polymeric substrate.

3. The method of manufacturing a flexible bioelectrode device according to claim 1, wherein each respective electrode comprises a conductive substrate.

4. The method of manufacturing a flexible bioelectrode device according to claim 1, wherein each respective electrode comprises a sensing/stimulating interface.

5. A flexible bioelectrode device comprising:
an electrically connected internal electrical circuit and wire array comprising:
an internal electrical circuit, the internal electrical circuit comprising a connector;
a wire array, the wire array comprising:
one or more terminal end(s), each one or more terminal end(s) terminating at a respective electrode; and
one or more distal end(s), each one or more distal end(s) electrically connected to the connector; and
a unibody polymeric body; and
wherein the electrically connected internal electrical circuit and wire array is at least partially encapsulated by the unibody polymeric body.

6. The flexible bioelectrode device according to claim 5, wherein the unibody polymeric body comprises a thermoplastic polymer selected from the group consisting of cellulose, cellulose derivatives, cyclic transparent optical polymer, parylene, polyamide (Nylon), polybutylene terephthalate, polycarbonate, polyester, polyethylene, polyethylene terephthalate, polyethylenimine, polylactic acid (PLA), polymethylmethacrylate, polypropylene, polystyrene, polyvinyl alcohol (PVA), styrene-ethylene-butylenestyrene, and thermoplastic polyurethane.

7. The flexible bioelectrode device according to claim 5, wherein the unibody polymeric body comprises a thermosetting polymer selected from the group consisting of latex, polychloroprene, polydimethylsiloxane (PDMS, silicone), polyimide (Kapton), and polyurethane.

8. The flexible bioelectrode device according to claim 5, wherein the wire array is fabricated on a polymeric film and wherein the polymeric film is permeable to a fluid prepolymer or polymer.

9. The flexible bioelectrode device according to claim 5, wherein each electrode comprises a sensing/stimulating interface and conductive substrate, surrounded by an insulating base.

10. The flexible bioelectrode device according to claim 9, further including a conductive gel, conductive liquid, or adhesive layer applied to an outer exposed surface of each sensing/stimulating interface of each electrode.

11. A method of manufacturing a flexible bioelectrode device, the method comprising the steps of:
preparing a polymeric film;
attaching the polymeric film to a fabrication bed;
applying a wire array, conductive substrate and a sensing/stimulating interface on the conductive substrate, to a surface of the polymeric film in a predetermined configuration to obtain a printed wire array thereon, wherein the conductive substrate and a sensing/stimulating interface combine to form a sensing electrode and the polymeric film supports the wire array in position as they are being fabricated in a desired configuration;
preparing an open mould of polymeric material and applying a non-stick substance onto the mould surface, wherein the open mould includes slots or recesses to receive a sensing electrode at predetermined locations;
placing a removable spacer into the slots or recesses;
aligning and positioning the polymer film with the fabricated electrodes and wires within the mould and inserting the fabricated electrodes into a corresponding recess above each filler or spacer;
aligning a connector terminal with terminal ends of the wire array and connecting to the wires;
preparing a fluid prepolymer and pouring the prepolymer over the open mould including the polymer film with the wires and electrodes, wherein the polymeric film allows the fluid prepolymer to permeate through the film when the polymer film is in contact with the fluid prepolymer thereby allowing the fluid prepolymer to surround the wire array on all sides;
allowing the fluid prepolymer to set or cure in the mould wherein the wire array is are embedded within the cured polymer;
removal of the set polymer from the mould;
applying conductive gel, liquid or adhesive to the exposed sensing/stimulating interface of the electrodes;
wherein the wire array is embedded within the cured polymer and electrode contact surfaces exposed so that the polymer body can flex and secure the wire array from being damaged.

12. The method of claim 11, wherein the fluid prepolymer is polydimethylsiloxane.

13. The method of claim 11, wherein the non-stick substance is polytetrafluoroethylene (PTFE).

14. The method of claim 11, wherein the polymer is selected from the group consisting of cellulose, cellulose derivatives, cyclic transparent optical polymer, parylene, polyamide (Nylon), polybutylene terephthalate, polycarbonate, polyester, polyethylene, polyethylene terephthalate, polyethylenimine, polylactic acid (PLA), polymethylmethacrylate, polypropylene, polystyrene, polyvinyl alcohol (PVA), styrene-ethylene-butylenestyrene, thermoplastic polyurethane, latex, polychloroprene, polydimethylsiloxane (PDMS, silicone), polyimide (Kapton), and thermosetting polyurethane.

15. A flexible bioelectrode device produced from the method of claim 11 comprising:
an electrically connected internal electrical circuit and wire array comprising:
an internal electrical circuit, the internal electrical circuit comprising a connector;
a wire array, the wire array comprising:
one or more terminal end(s), each one or more terminal end(s) terminating at a respective electrode; and
one or more distal end(s), each one or more distal end(s) electrically connected to the connector; and
a polymeric body formed by the cured fluid prepolymer; and
wherein the electrically connected internal electrical circuit and wire array is at least partially encapsulated by the polymeric body so that the polymeric body is able to flex and substantially prevent the wire array from being damaged by delamination.
